(19) United States
(12) Patent Application Publication
(10) Pub. No.: US 2026/0263403 A1
(43) Pub. Date: Sep. 10, 2026

(54) COMPOSITIONS AND METHODS FOR TREATING TRAUMATIC BRAIN INJURY

(71) Applicant: THE CHILDREN'S HOSPITAL OF PHILADELPHIA, Philadelphia, PA (US)

(72) Inventor: Akiva S. Cohen, Bala Cynwyd, PA (US)

(21) Appl. No.: 19/473,704

(22) PCT Filed: May 17, 2024
(86) PCT No.: PCT/US2024/029816

§ 371 Date: Oct. 8, 2025

Related U.S. Application Data
(60) Provisional application No. 63/467,735, filed on May 19, 2023.

(51) Int. Cl. A61K 31/198; A61K 31/225; A61P 25/06; A61P 25/20

(52) U.S. Cl. CPC A61K 31/198; A61K 31/225; A61P 25/06; A61P 25/20

(57) ABSTRACT

Compositions and methods are provided for the treatment of pathologies associated with traumatic brain injury.

This application claims priority under 35 U.S.C. § 119(e) to U.S. Provisional Patent Application No. 63/467,735, filed on May 19, 2023. The foregoing application is incorporated by reference herein.

This invention was made with government support under grant nos. NS069629 and HD059288 awarded by the National Institutes of Health. The government has certain rights in the invention.

### FIELD OF THE INVENTION

The present invention relates to the alleviation of pathology associated with traumatic brain injury.

### BACKGROUND OF THE INVENTION

Mild traumatic brain injuries (mTBI), including concussion, are common injuries experienced by adolescents and young adults (Bryan, et al. (2016) Pediatrics 138(1):e20154635). Over the past decade, significant advances in the knowledge and care of concussed adolescents and young adults have occurred. While traditionally, a passive strategy of cognitive and physical rest was recommended as the sole treatment modality for concussion (Halstead, et al. (2010) Pediatrics 126(3):597-615), over this time period, several active rehabilitation strategies have shown efficacy, including aerobic exercise therapy (Ledd, et al. (2019) JAMA Pediatr., 173(4):319-325; Leddy, et al. (2021) Lancet Child Adolesc. Health 5(11):792-799) and visio-vestibular rehabilitation (Kontos, et al. (2021) J. Pediatr., 239:193-199; Storey, et al. (2018) J. Neurol. Phys. Ther., 42(3):134-141). However, these therapeutics are time and labor intensive, and require specialized monitoring and prescription from a concussion specialist. In spite of these important advances, a targeted pharmacologic intervention to hasten recovery of concussed youth and young adults has yet to be identified. Specifically, the vast majority of medications prescribed for adolescent and young adult concussion focus on symptom amelioration (such as over-the-counter analgesics and anti-emetics (Mannix, et al. (2019) J. Neurotrauma 36(20):2886-2894)), without necessarily targeting the underlying pathophysiology that leads to concussion symptoms (Giza, et al. (2014) Neurosurgery 75(04):S24-S33). New methods of targeted treatment for the inhibition and/or prevention of the underlying pathology associated with traumatic brain injury are needed.

### SUMMARY OF THE INVENTION

In accordance with the instant invention, methods for inhibiting, reducing, treating, and/or preventing in a subject a pathology or symptom associated with traumatic brain injury are provided. In certain embodiments, the method comprises administering to the subject at least one branched chain amino acid (BCAA), particularly all three branched chain amino acids. In certain embodiments, the BCAAs are administered orally. In certain embodiments, the traumatic brain injury is an acute, mild traumatic brain injury or a concussion. In certain embodiments, the subject is a youth, adolescent or young adult. In certain embodiments, the pathology is headache, decreased physical activity, and/or decreased sleep efficiency and/or sleep time.

In accordance with the instant invention, methods for inhibiting, reducing, treating, and/or preventing a headache in a subject are provided. In certain embodiments, the method comprises administering to the subject at least one branched chain amino acid (BCAA), particularly all three branched chain amino acids. In certain embodiments, the BCAAs are administered orally. In certain embodiments, the subject has a traumatic brain injury. In certain embodiments, the traumatic brain injury is an acute, mild traumatic brain injury or a concussion. In certain embodiments, the subject is a youth, adolescent or young adult. In certain embodiments, the presence (or frequency), duration, and/or severity of the headache is reduced.

In accordance with another aspect of the instant invention, compositions comprising the branched chain amino acids and, optionally, at least one pharmaceutically acceptable carrier are provided. The compositions may be in an orally acceptable form such as a liquid or powder (e.g., lyophilized) form.

### BRIEF DESCRIPTIONS OF THE DRAWING

FIG. 1 provides a flow diagram of allocation and follow-up.

FIGS. 2A-2D provide graphs of the adjusted analysis (comprised of cumulative study dose received over the study period) of a neurocognitive testing battery of four measures following injury, adjusted for age and repeated measures. Solid line is trend and shaded area is 95% confidence interval. FIG. 2A shows the changes in the processing speed subtest over the study period. FIG. 2B shows the changes in the attention subtest. FIG. 2C shows the changes in the working memory subtest. FIG. 2D shows the changes in the visual learning accuracy subtest.

FIGS. 3A-3C provide graphs of the adjusted analysis (comprised of cumulative study dose received over the study period, adjusting for age and repeated measures) of overall concussion symptoms (including headache), physical, and cognitive activity change. Solid line is trend and shaded area is 95% confidence interval. FIG. 3A shows the changes in the total symptom score. FIG. 3B shows the changes in reported physical activity. FIG. 3C shows the changes in reported cognitive activity.

FIG. 4 provides a graph of the adjusted analysis of headache reported with total BCAA consumed.

### DETAILED DESCRIPTION OF THE INVENTION

Herein, the efficacy, tolerability, and safety of varied doses of oral branched chain amino acid (BCAA) supplementation in a group of concussed adolescents and young adults is provided. Specifically, randomized, double-blind, placebo-controlled trial was performed on participants ages 11-34 presenting with concussion to the outpatient clinics (sports medicine and primary care), urgent care, and emergency departments of a tertiary care pediatric children's hospital and an urban tertiary care adult hospital. Participants were randomized to one of five study arms (placebo and 15 g, 30 g, 45 g, and 54 g BCAA treatment daily) and followed for 21 days after enrollment. Outcome measures included daily computerized neurocognitive tests (processing speed, attention, visual learning, and working memory), symptom score, self-reported physical and cognitive activity, sleep/wake alterations, treatment compliance, and adverse events.

In total, 42 participants were randomized, 38 of whom provided analyzable data. A significant reduction in both total symptom score (p-value for trend=0.0034), particularly headache, was found along with improvement in physical activity (p-value for trend=0.005) by study dose, with increased sleep efficiency (p-value=0.0360) and total sleep time (p-value=0.0420). There were no significant differences in any of the four neurocognitive testing batteries or cognitive activity. There was also a non-significant decrease in average reported compliance by arm. No serious adverse events in the trial were reported with only 8 of 38 participants reporting a mild or moderate adverse event. Notably only 2 reported mild adverse events (both gastrointestinal) occurred in the highest (45 g and 54 g) BCAA arms.

This study provides the first demonstration of efficacy of BCAAs in concussed adolescents and young adults—specifically, a dose response effect in reducing concussion symptoms (specifically headache), improving post-injury physical activity, and improvements in sleep with those treated with higher total doses of BCAAs.

The instant invention provides a BCAA rich composition, particularly a liquid consumable, for treating a traumatic brain injury, particularly a mild traumatic brain injury such as a concussion. In certain embodiments, the composition comprises valine, leucine, and isoleucine in approximately equivalent amounts (e.g., a 1:1:1 ratio or ±5%). The amount of BCAA may be determined, for example, by weight or molar amount. While the BCAAs will often be present in equivalent amounts, the compositions of the instant invention may comprise excess amounts of one or two of the BCAAs. For example, the composition may comprise up to ten times, up to five times, up to three times, or up to two times excess of one or two amino acids branched amino acid compared to another. For example, the composition may comprise excess valine.

Branched-chain amino acids are amino acids that have a fork or branch in the side chain. Branched chain amino acids include leucine, isoleucine and valine and precursors or analogs thereof. BCAAs may be administered in their free forms or salts thereof, as dipeptides, tripeptides, polypeptides (e.g., from about 2 to about 10 amino acids), and/or BCAA-rich proteins (e.g., proteins comprising at least 25%, at least 50%, or at least 75% or more BCAA content). In certain embodiments, dipeptides, tripeptides and polypeptides may include two or more BCAAs. Where non-BCAAs are included in a dipeptide, tripeptide, or polypeptide, the non-BCAAs may be any amino acid, particularly alanine and/or glycine. Examples of dipeptides include, without limitation: isoleucyl-leucine, leucyl-alanine, alanyl-leucine, alanyl-isoleucine, alanyl-valine, glycyl-leucine, glycyl-isoleucine, and glycyl-valine.

Leucine precursors, such as pyruvate, and metabolites, such as β-hydroxy-β-methylbutyrate and α-ketoisocaproate, exhibit properties similar to those of leucine. These compounds may be administered as BCAAs as they are converted into the above-mentioned BCAA in vivo.

In certain embodiments, the composition comprises independently from about 1 mg/ml to about 50 mg/ml, from about 5 mg/ml to about 25 mg/ml, from about 10 mg/ml to about 20 mg/ml, from about 13 mg/ml to about 20 mg/ml, or about 16 mg/ml of each of the three BCAA.

In certain embodiments, at least about 40 g, at least about 45 g, at least about 50 g, at least about 54 g, at least about 60 g, at least about 70 g, at least about 75 g, at least about 80 g, at least about 85 g, at least about 90 g, at least about 95 g, at least about 100 g, or more of BCAAs are administered to the subject per day. In certain embodiments, about 40 g to about 100 g of BCAAs are administered daily, particularly about 50 g to about 100 g, about 60 g to about 100 g, about 60 g to about 75 g, or about 60 g. Taking the subject's weight into account, at least about 40 g/70 kg, at least about 50 g/70 kg, at least about 60 g/70 kg, at least about 70 g/70 kg, or more of BCAAs are administered to the subject per day. In certain embodiments, about 40 g/70 kg to about 100 g/70 kg of BCAAs are administered daily, particularly about 50 g/70 kg to about 100 g/70 kg, about 60 g/70 kg to about 100 g/70 kg, about 60 g/70 kg to about 75 g/70 kg, or about 60 g/70 kg. The BCAAs may be administered in more than one dosage to reach the daily goal (e.g., administered twice, three times, four times or more daily).

In certain embodiments, the subject is administered at least 1000 g, at least 1250 g, at least 1500 g, at least 1750 g, at least 2000 g, at least 2250 g, at least 2500 g, at least 2750 g, or at least 3000 g of BCAAs during the treatment period. In certain embodiments, the treatment period is at least 7 days, at least 10 days, or at least two weeks. In certain embodiments, the treatment period is two weeks or less, three weeks or less, four weeks or less, or five weeks or less. In certain embodiments, the BCAAs are administered evenly or nearly evenly across the treatment period (e.g., with the same or similar daily dose). In certain embodiments, the BCAAs are administered on consecutive days (e.g., daily).

Compositions comprising large quantities of branched chain amino acids are not palatable to many subjects, particularly humans. Accordingly, compositions of the instant invention comprise at least one flavor masking agent, particularly a bitter taste receptor blocker. In certain embodiments, the flavor masking agent is a salt of an organic acid. Examples of organic acids include, without limitation, gluconic acid and lactic acid. Suitable salts include, by way of example, calcium lactate, magnesium lactate, sodium lactate, calcium gluconate, magnesium gluconate, and sodium gluconate. In certain embodiments, the flavor masking agent is sodium gluconate.

In certain embodiments, the flavor masking agent is present in the composition at about 5 g, at about 10 g, at about 15 g, at about 20 g, or more per 30 g of BCAAs. In certain embodiments, the composition comprises at least about 50 mM, at least about 75 mM, at least about 100 mM, or more of the flavor masking agent.

The compositions may also comprise at least one sweetener. Any natural or artificial sweetener may be used. Examples of sweeteners include, without limitation, sucrose, fructose, maltose, dextrose, sucralose, aspartame, saccharin, and the like. In certain embodiments, the sweetener is sucralose.

In certain embodiments, the sweetener is present in the composition at about 2 g, at about 5 g, at about 10 g, at about 15 g, or more per 30 g of BCAAs. In certain embodiments, the composition comprises at least about 10 mM, at least about 20 mM, at least about 35 mM, or more of the sweetener.

The compositions may also comprise at least one flavoring agent. Any natural or synthetic flavor agent can be used in the present invention. One or more flavoring agents may be used to enhance the palatability of the compositions. The flavoring agent may be an emulsion, concentrate, aqueous- or oil-soluble liquid, dry powder, or a combination thereof. In certain embodiments, the flavor agent is a concentrate or powder. In certain embodiments, the flavoring agent is a fruit flavor. In certain embodiments, the flavor agent is a concentrate powder (e.g., unsweetened tropical punch KoolAid® powder).

The compositions of the instant invention may also comprise acetate. Acetate may be present as acetic acid or a pharmaceutically acceptable salt thereof, such as calcium acetate. In certain embodiments, the acetate is present in a hydrophobic form, such as glyceryl triacetate (GTA; the acetate triester of glycerol). It has been shown that GTA can be used as a dietary supplement to increase acetate levels in the brain by over 15-fold within one hour of administration (Mathew et al. (2005) J. Pharmacol. Exp. Ther., 315:297-303). In certain embodiments, about 0.5 mg/kg to about 100 mg/kg of GTA are administered daily, particularly about 0.5 mg/kg to about 10 mg/kg, about 2.5 mg/kg to about 7.5 mg/kg, or about 5 mg/kg.

In accordance with another aspect of the instant invention, methods for treating, preventing, and/or inhibiting a pathology (e.g., symptom) associated with a traumatic brain injury in a subject are provided. The methods comprise administering branched chain amino acids to a subject who has sustained a traumatic brain injury (e.g., by administering a BCAA composition as described herein to the subject). The methods of the instant invention may further comprise examining/diagnosing the subject for a pathology (e.g., symptom) after the traumatic brain injury and prior to treatment. The methods may also further comprise examining/diagnosing the subject for the pathology (e.g., symptom) after the traumatic brain injury and after treatment. Treatment with the BCAAs may be stopped once the pathology (e.g., symptom) has been resolved.

In accordance with another aspect of the instant invention, methods for treating, preventing, and/or inhibiting a headache in a subject are provided. The methods comprise administering branched chain amino acids to a subject in need thereof (e.g., by administering a BCAA composition as described herein to a subject with a headache). In certain embodiments, the subject has a traumatic brain injury. who has sustained a traumatic brain injury. The methods of the instant invention may further comprise examining/diagnosing the subject for a headache (e.g., after a traumatic brain injury and prior to treatment). The methods may also further comprise examining/diagnosing the subject for the headache (e.g., after a traumatic brain injury) after treatment. Treatment with the BCAAs may be stopped once the headache has been resolved.

In certain embodiments, the subject is a youth, adolescent, or young adult. In certain embodiment, the subject is less than about 40 years old or less than about 35 years old. In certain embodiments, the subject is less than 25 years old, less than 21 years old, or less than 18 years old. In certain embodiments, the subject is more than about 10 years old.

As used herein, “traumatic brain injury” or “TBI” refers to an acquired brain injury or a head injury, when a trauma causes damage to the brain. Trauma includes, e.g., post-head trauma, impact trauma, and other traumas to the head such as, for example, traumas caused by accidents and/or sports injuries and concussive injuries. In certain embodiments, the trauma is an external, physical force. In certain embodiments, the traumatic brain injury is a closed head injury. In certain embodiments, the traumatic brain injury is acute. In certain embodiments, the traumatic brain injury is a concussion.

The damage can be focal (confined to one area of the brain) or diffuse (involving more than one area of the brain). Clinically, traumatic brain injury can be rated as mild, moderate or severe based on TBI variables that include duration of loss of consciousness (LOC), Glasgow Coma Score (GCS; e.g., mild 13-15; moderate=9-12; severe=≤8) and post traumatic stress amnesia (see, e.g., Levin et al. (1979) J. Nervous Mental Dis., 167:675-84; Holm et al. (2005) J. Rehabil. Med., 37:137-41). In certain embodiments, the TBI is mild.

In certain embodiments, the traumatic brain injury can be chronic, where the brain is subject to repeated traumatic injury to the brain. Generally, chronic traumatic brain injury is typically a mild to moderate form of closed brain injury repeatedly suffered by a subject (e.g., athlete), resulting in increased incidence of impaired motor, cognitive, and/or behavioral impairments months to years following the traumatic brain injuring events.

In certain embodiments, the traumatic brain injury can result from a closed head injury. The closed head injury may be transient or prolonged. A “closed head injury” refers to a brain injury when the head suddenly and violently hits an object but the object does not break through the skull. In some embodiments, the closed head injury is a concussion or contusion. A concussion is a mild form of traumatic brain injury resulting in temporary impairment of neurological function and where there are generally no gross structural changes to the brain as the result of the condition. A contusion is a distinct area of swollen brain tissue mixed with blood released from broken blood vessels. A contusion can also occur in response to shaking of the brain back and forth within the confines of the skull, an injury referred to as “contrecoup.” As used herein, a closed head injury refers to an injury due to an external, physical trauma and does not encompass brain injury resulting from “internal” forces such as ischemia/reperfusion and stroke.

Pathologies (e.g., symptoms) of traumatic brain injury that can be treated include, without limitation, headache (e.g., secondary headache, post-traumatic headache, chronic post-traumatic headache, e.g., headache within 7 days of brain injury), nausea, balance problems, sleep disorders (e.g., sleeping more than normal), drowsiness, fatigue, feeling slowed down, feeling mentally foggy, difficulty concentrating, decreased physical activity, and decreased cognitive activity. Examples of sleep disorders associated with a traumatic brain injury include, without limitation, insomnia (e.g., difficulty with falling asleep and/or staying asleep), fatigue, drowsiness, sleep apnea, narcolepsy, and/or sleepiness (e.g., excessive daytime sleepiness). Sleep disorders may be analyzed by studying changes in bedtime, wake time, total sleep time, sleep efficiency, wake after sleep onset, number and length of awakenings, activity, and movement/sleep fragmentation. Sleep disorders may be diagnosed any means such as a sleep study, polysomnography, multiple sleep latency testing, maintenance of wakefulness testing, and/or actigraphy.

In certain embodiments, the symptom treated by the method of the instant invention is headache. In certain embodiments, the method comprises administering branched chain amino acids to a subject to reduce the presence, severity, length or duration, and/or frequency of headaches experienced by the subject. The method may further comprise monitoring (e.g., one or more times) the subject for headaches after administration of the branched chain amino acids. For example, the subject may be monitored for headaches after the completion of a regimen of doses of branched chain amino acids or portion thereof.

In certain embodiments, the pathology treated by the method of the instant invention is decreased physical activity. In certain embodiments, the method comprises administering branched chain amino acids to a subject to increase physical activity (e.g., return to physical activity levels prior to the traumatic brain injury) by the subject. The method may further comprise monitoring (e.g., one or more times) the subject for physical activity after administration of the branched chain amino acids. For example, the subject may be monitored for physical activity after the completion of a regimen of doses of branched chain amino acids or portion thereof.

In certain embodiments, the pathology treated by the method of the instant invention is decreased sleep efficiency and/or sleep time. In certain embodiments, the method comprises administering branched chain amino acids to a subject to increase sleep efficiency and/or sleep time (e.g., return to sleep efficiency and/or sleep time experienced prior to the traumatic brain injury) by the subject. The method may further comprise monitoring (e.g., one or more times) the subject for sleep efficiency and/or sleep time after administration of the branched chain amino acids. For example, the subject may be monitored for sleep efficiency and/or sleep time after the completion of a regimen of doses of branched chain amino acids or portion thereof.

In certain embodiments, methods of the instant invention treat more than one pathology. In certain embodiments, the pathologies treated are any combination of headache, decreased physical activity, and decreased sleep efficiency and/or sleep time.

As stated above, the methods of the instant invention comprise administering branched chain amino acids to a subject in need thereof. In certain embodiments, the subject has sustained a traumatic brain injury. In certain embodiments, the subject has a headache. The BCAAs may be administered before, during, and/or after the traumatic brain injury. For example, the subject may be administered the BCAAs prior to sustaining a traumatic brain injury (e.g., prior to participating in an activity with an increased likelihood of sustaining a traumatic brain injury such as participating in a contact sport (e.g., hockey, football, rugby, etc.). The subject may be administered BCAA therapy after sustaining the injury.

The BCAAs may be administered to the subject by any means. In certain embodiments, the BCAAs are administered orally or in a liquid consumable (e.g., the palatable/drinkable compositions described herein). The BCAAs administered to the subject may include one, two, or all three of valine, leucine, and isoleucine. In certain embodiments, valine, leucine, and isoleucine are administered in approximately equivalent amounts (e.g., a 1:1:1 ratio or 5%). The amount of BCAA may be determined, for example, by weight or molar amount. While the BCAAs will often be administered in equivalent amounts, excess amounts of one or two of the BCAAs may be administered. For example, up to ten times, up to five times, up to three times, or up to two times excess of one or two amino acids compared to another may be administered. For example, the excess valine compared to leucine and/or isoleucine may be administered.

As explained hereinabove, the BCAAs may be administered in their free forms or salts thereof, as dipeptides, tripeptides, polypeptides (e.g., from about 2 to about 10 amino acids), and/or BCAA-rich proteins (e.g., proteins comprising at least 25%, at least 50%, or at least 75% or more BCAA content). The BCAAs may be isolated. In certain embodiments, dipeptides, tripeptides and polypeptides may include two or more BCAAs. Where non-BCAAs are included in a dipeptide, tripeptide, or polypeptide, the non-BCAAs may be any amino acid, particularly alanine and/or glycine. Examples of dipeptides include, without limitation: isoleucyl-leucine, leucyl-alanine, alanyl-leucine, alanyl-isoleucine, alanyl-valine, glycyl-leucine, glycyl-isoleucine, and glycyl-valine.

Leucine precursors, such as pyruvate, and metabolites, such as β-hydroxy-β-methylbutyrate and α-ketoisocaproate, exhibit properties similar to those of leucine. These compounds may be administered as BCAAs as they are converted into the above-mentioned BCAA in vivo.

In certain embodiments, at least about 40 g, at least about 45 g, at least about 50 g, at least about 54 g, at least about 60 g, at least about 70 g, at least about 75 g, at least about 80 g, at least about 85 g, at least about 90 g, at least about 95 g, at least about 100 g, or more of BCAAs are administered to the subject per day. In certain embodiments, about 40 g to about 100 g of BCAAs are administered daily, particularly about 50 g to about 100 g, about 60 g to about 100 g, about 60 g to about 75 g, or about 60 g. Taking the subject's weight into account, at least about 40 g/70 kg, at least about 50 g/70 kg, at least about 60 g/70 kg, at least about 70 g/70 kg, or more of BCAAs are administered to the subject per day. In certain embodiments, about 40 g/70 kg to about 100 g/70 kg of BCAAs are administered daily, particularly about 50 g/70 kg to about 100 g/70 kg, about 60 g/70 kg to about 100 g/70 kg, about 60 g/70 kg to about 75 g/70 kg, or about 60 g/70 kg. The BCAAs may be administered in more than one dosage to reach the daily goal (e.g., administered twice, three times, four times or more daily).

In certain embodiments, the subject is administered at least 1000 g, at least 1250 g, at least 1500 g, at least 1750 g, at least 2000 g, at least 2250 g, at least 2500 g, at least 2750 g, or at least 3000 g of BCAAs during the treatment period. In certain embodiments, the treatment period is at least 7 days, at least 10 days, or at least two weeks. In certain embodiments, the treatment period is two weeks or less, three weeks or less, four weeks or less, or five weeks or less. In certain embodiments, the BCAAs are administered evenly or nearly evenly across the treatment period (e.g., with the same or similar daily dose). In certain embodiments, the BCAAs are administered on consecutive days (e.g., daily).

In certain embodiments, the BCAAs are administered immediately or soon after the traumatic brain injury event. For example, the BCAAs may be administered at least within a month of injury, within two weeks of injury, within about the first 2, 3, 4 or 7 days after injury, within about the first day after injury, or within about the first hour after injury. In certain embodiments, the subject is administered BCAA therapy within 72 hours, within 48 hours, or within 24 hours of the traumatic brain injury. In certain embodiments, the BCAAs are administered within about the first 2 days of the injury. The BCAAs may be administered continually (e.g., every day) after the injury for at least one week, particularly at least two weeks, at least three weeks, at least four weeks or more.

The methods of the instant invention may also comprise administering acetate. Acetate may be administered as acetic acid or a pharmaceutically acceptable salt thereof, such as calcium acetate. In certain embodiments, the acetate is present in a hydrophobic form, such as glyceryl triacetate (GTA; the acetate triester of glycerol). In certain embodiments, about 0.5 mg/kg to about 100 mg/kg of GTA are administered daily, particularly about 0.5 mg/kg to about 10 mg/kg, about 2.5 mg/kg to about 7.5 mg/kg, or about 5 mg/kg.

The agents of the instant invention used to treat, inhibit, reduce, and/or prevent of the symptoms and/or pathology associated with traumatic brain injury may be administered to a patient orally, such as a liquid consumable or pharmaceutical preparation. The term “patient” as used herein refers to human or animal subjects.

The instant invention encompasses compositions comprising BCAAs and, optionally, at least one pharmaceutically acceptable carrier or other component as described hereinabove. As used herein, “pharmaceutically acceptable carrier” includes any and all solvents, dispersion media and the like which may be appropriate for the desired route of administration of the pharmaceutical preparation. The use of such media for pharmaceutically active substances is known in the art. Except insofar as any conventional media or agent is incompatible with the molecules to be administered, its use in the pharmaceutical preparation is contemplated.

The compositions comprising at least one of the agents of the instant invention may be conveniently formulated for administration with a pharmaceutically acceptable carrier. Solubility limits of the agents within the particular pharmaceutically acceptable carrier may be easily determined by one skilled in the art.

Compositions of the instant invention may be administered orally. The pharmaceutical composition of the present invention can be prepared, for example, in liquid form (including concentrated liquid form requiring dilution (e.g., with water) prior to consumption), or can be in dried powder form (e.g., lyophilized for later reconstitution (e.g., with water)). Dosage forms for oral administration include, without limitation, tablets (e.g., coated and uncoated, chewable), sustained release capsules, gelatin capsules (e.g., soft or hard), solutions, drinks, concentrates, emulsions, suspensions, syrups, elixirs, and powders/granules (e.g., reconstitutable or dispersible).

The BCAAs of the instant invention may also be administered nasally (e.g., via nasal pump, nasal spray, nasal drops, etc.). For example, a composition comprising the BCAAs can be administered as an aerosol formulation which contains the BCAAs in dissolved, suspended or emulsified form in a propellant or a mixture of solvent and propellant. The aerosolized formulation is then administered through the respiratory system or nasal passages. An aerosol formulation used for nasal administration may be an aqueous solution designed to be administered to the nasal passages in drops or sprays. The nasal solutions may also be administered without propellant—e.g., via nasal drops, spray or mist (e.g., an atomized spray). Nasal solutions are generally prepared to be similar to nasal secretions and are generally isotonic and slightly buffered to maintain a pH of about 5.5 to about 6.5, although pH values outside of this range can additionally be used (e.g., a pH of about 5 to about 8). Antimicrobial agents or preservatives can also be included in the formulation.

Pharmaceutical compositions containing agents of the present invention as the active ingredient in intimate admixture with a pharmaceutically acceptable carrier can be prepared according to conventional pharmaceutical compounding techniques. The carrier may take a wide variety of forms depending on the form of preparation desired for administration, e.g., oral.

The compounds/compositions of the instant invention may also be encapsulated within a protective material (e.g., one that is polymeric in nature or one which is lipid-based (e.g., liposomes or micelles)). The polymeric drug delivery system may be colloidal or non-colloidal in nature and may be composed of biodegradable or non-biodegradable polymeric materials. Colloidal polymeric encapsulation structures include, without limitation, microparticles, microspheres, nanoparticles, and nanospheres, block copolymer micelles, and the like. In certain embodiments, the compounds and/or compositions of the instant invention may be formulated within a nanomaterial carrier. Suitable nanomaterial carriers are known to those skilled in the art. In certain embodiments, the nanomaterial carrier is a nanoparticle or a nanosphere. Nanoparticles or nanospheres typically range from about 1 to 1,000 nanometers (nm), particularly from about 50 to 300 nm. The term “nanosphere” refers to a type of nanoparticle that is approximately spherical in shape. The nanosphere may have a hollow core within which one or more compounds can be placed.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art.

Dosage units may be proportionately increased or decreased based on the weight of the patient. Appropriate concentrations for alleviation of a particular pathological condition may be determined by dosage concentration curve calculations, as known in the art.

In accordance with the present invention, the appropriate dosage unit for the administration of compositions of the instant invention may be determined by evaluating the toxicity of the molecules or cells in animal models. Various concentrations of the agents in pharmaceutical preparations may be administered to mice, and the minimal and maximal dosages may be determined based on the beneficial results and side effects observed as a result of the treatment. Appropriate dosage unit may also be determined by assessing the efficacy of the treatment in combination with other standard drugs. The dosage units may be determined individually or in combination with each treatment according to the effect detected.

The compositions of the instant invention may be administered at appropriate intervals, for example, at least twice a day or more until the pathological symptoms are reduced or alleviated, after which the dosage may be reduced to a maintenance level. The appropriate interval in a particular case would normally depend on the condition of the patient.

In certain embodiments, the composition(s) of the instant invention are administered immediately or soon after the traumatic brain injury event. For example, the pharmaceutical preparation is administered at least within a month of injury, within two weeks of injury, within about the first 7 days after injury, within about the first day after injury, within about the first hour after injury.

The compositions of the instant invention may be administered continually (e.g., every day) after the injury for at least one week, particularly at least two weeks, at least three weeks, at least four weeks or more.

In general, the compositions of the instant invention may contain other components in amounts that do not detract from the preparation of effective safe formulations. The compositions of the instant invention may further comprise at least one preservative, stabilizer, carriers, excipients, and/or antibiotic. The compositions and methods of the instant invention may also be combined with other compositions and methods for the treatment of the symptoms and/or pathology of a traumatic brain injury.

### Definitions

The singular forms “a,” “an,” and “the” include plural referents unless the context clearly dictates otherwise.

As used herein, “cognitive impairment” refers to an acquired deficit in at least one of the following: memory function, problem solving, orientation, and abstraction. The deficiency typically impinges on an individual's ability to function independently.

The term “substantially pure” refers to a preparation comprising at least 50-60% by weight of a given material (e.g., nucleic acid, oligonucleotide, protein, etc.). More preferably, the preparation comprises at least 75% by weight, and most preferably 90-95% by weight of the given compound. Purity is measured by methods appropriate for the given compound (e.g., chromatographic methods, agarose or polyacrylamide gel electrophoresis, HPLC analysis, and the like).

The terms “isolated” is not meant to exclude artificial or synthetic mixtures with other compounds or materials, or the presence of impurities that do not interfere with the fundamental activity, and that may be present, for example, due to incomplete purification, or the addition of stabilizers.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, preservative, antioxidant, solubilizer, emulsifier, adjuvant, excipient, bulking substances, auxiliary agent or vehicle with which an active agent of the present invention is administered. Pharmaceutically acceptable carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. Suitable pharmaceutical carriers are described, for example, in “Remington's Pharmaceutical Sciences” by E. W. Martin.

The term “pathology” refers to any deviation from a healthy or normal condition, such as a disease, disorder, syndrome, or any abnormal medical condition.

The term “treat” as used herein refers to any type of treatment that imparts a benefit to a patient suffering from an injury (e.g., TBI), including improvement in the condition of the patient (e.g., in one or more symptoms), delay in the progression of the condition, etc.

As used herein, the term “prevent” refers to the prophylactic treatment of a subject who is at risk of developing a condition and/or sustaining an injury (e.g., TBI or post-traumatic concussion symptoms) resulting in a decrease in the probability that the subject will develop conditions associated with the injury.

A “therapeutically effective amount” of a compound or a pharmaceutical composition refers to an amount effective to prevent, inhibit, or treat a particular injury and/or the symptoms thereof. For example, “therapeutically effective amount” may refer to an amount sufficient to modulate the pathology associated traumatic brain injury in a patient.

As used herein, the term “subject” refers to an animal, particularly a mammal, particularly a human.

The following example describes illustrative methods of practicing the instant invention and is not intended to limit the scope of the invention in any way.

### EXAMPLE

The three branched chain amino acids (BCAAs)—valine, leucine, and isoleucine—are specifically and significantly reduced following TBI, with their depletion possibly the result of their role in both the production of neurotransmitters and in cellular metabolism, which is transiently elevated after injury followed by a protracted duration of reduction (Cole, et al. (2010) Proc. Natl. Acad. Sci., 107(1):366-371). Following BCAA supplementation, injured mice demonstrate restoration of excitatory/inhibitory (EI) balance in the hippocampus, improvement in cognitive activity, and mitigation of persistent sleep-wake deficits (Elkind, et al. (2015) Front Neurol., 6:73; Lim, et al. (2013) Sci. Transl. Med., 5(215):215ra173). In trials of adults with severe TBI, BCAA levels have been found to correlate with measures of clinical severity (such as intracranial pressure (Vuille-Dit-Bille, et al. (2012) Amino Acids 43(3):1287-1296)), with BCAA supplementation leading to moderate clinical improvements involving disability and cognitive function in the severely injured population (Aquilani, et al. (2008) Arch. Phys. Med. Rehabil., 89(9):1642-1647; Aquilani, et al. (2005) Arch. Phys. Med. Rehabil., 86(9):1729-1735). Studies of BCAAs in the mTBI population, specifically among pediatric patients, are much more limited. A single study of 18 (primarily adult) mTBI participants showed BCAAs and their metabolites are decreased compared to healthy controls (Jeter, et al. (2013) J. Neurotrauma 30(8):671-679). Additionally, a study in veterans in the chronic phase of recovery from mTBI demonstrated preliminary efficacy for BCAA supplementation to improve both self-reported sleep and objective actigraphic markers of sleep (i.e., sleep onset latency, sleep efficiency, and wake after sleep onset (Elliott, et al. (2022) Front. Syst. Neurosci., 16:854874)). However, to date, no studies have evaluated either the efficacy or tolerability of BCAA supplementation in concussed adolescents and young adults shortly following the injury.

Herein, a randomized, double-blinded, placebo-controlled trial is provided which 1) evaluates the efficacy of BCAA supplementation in a group of concussed adolescents and young adults, with particular attention to neurocognitive performance, symptom (specifically headache) and activity changes, and sleep disturbances, and 2) determines the safety and tolerability of various doses of BCAA supplementation levels. A dose-response improvement in neurocognitive performance (particularly processing speed), symptom burden (including headache), activity level, and sleep disturbances in those receiving BCAA therapy compared to controls, with BCAAs being well-tolerated across a variety of doses (Evangeliou, et al. (2009) J. Child Neurol., 24(10):1268-1272; Richardson, et al. (2004) J. Clin. Psychiatry 65(1):92-96), was hypothesized.

### Methods

### Study Design and Participant Population

The reported trial was registered on ClinicalTrials.gov (identifier NCT01860404) prior to participant enrollment. Additionally, the study was a US Food and Drug Administration (FDA) regulated study (FDA IND 117570). Participants were enrolled as a convenience sample, based on availability of research team members, from the outpatient clinics (sports medicine and primary care), urgent care centers, and emergency departments of a tertiary care pediatric children's hospital and an urban tertiary care adult hospital, both with a level I trauma designations, from Jun. 25, 2014, through Dec. 5, 2020. Of note, in October 2016, after the enrollment of 12 analyzable subjects, the study was paused due to slow enrollment, and eligibility criteria were slightly modified in three domains (age limit, time from injury to presentation, and injury mechanism) as noted below. Study enrollment resumed in 2018, however slowed significantly again during the novel coronavirus pandemic in early 2020. Therefore, in late 2020, after randomizing 42 participants, given the ongoing length of the study to date and concurrent advances in concussion care, enrollment was halted to assess efficacy of the data.

Participants were eligible if they were between 11 and 34 years of age (initial lower age limit was 16 years, lowered following initial enrollment phase) and diagnosed with a concussion by the treating physician or advanced practice provider in alignment with the most recent International Consensus Concussion guidelines (McCrory, et al. (2013) Br. J. Sports Med., 47:250-258). Additional inclusion criteria included injury within the prior 72 hours (initial criteria was injury within 24 hours, broadened following the initial study phase), weight of at least 40 kg (given dosing considerations (Gijsman, et al. (2002) Psychopharmacology 160(2):192-197)), and a negative pregnancy test and acceptable contraception for study duration for post-menarchal females (due to animal models showing possible harmful effects of BCAA supplementation in utero (To, et al. (2020) Int. J. Environ. Res. Public Health 17(7):2445)). Initial injury mechanism was limited to sport- and recreation-related injuries; however, this was broadened to include all injury mechanisms after the initial enrollment phase. Exclusion criteria included signs of a moderate or severe TBI (including witnessed seizure or admission to an intensive care unit), prior concussion within the preceding 90 days, history of maple syrup urine disease or family history of maple syrup urine disease, active prescription of neurologic or psychoactive medications, and allergy to red dye #40 or sucralose. Following screening, all participants (for those 18 years and older) or parents/guardians (for those younger than 18 years) provided written informed consent; child assent was also obtained for participants younger than 18 years of age.

### Randomization and Blinding

Following enrollment and completion of informed consent, participants were randomized into one of five groups (placebo and four BCAA dosing groups, as noted below), in a schema of equal allocation, stratified by sex, which was generated by the study biostatistician apriori. A randomization module was programmed to generate a unique kit number used to identify each participant. The scheme was provided to the Investigational Drug Service (IDS) at the University of Pennsylvania prior to study initiation, where study kits were ultimately prepared. Both participant and all study investigators were blinded to treatment assignment; only the Database Administrator as well as the University of Pennsylvania Pharmacist had access to the link between kit number and treatment arm. Color and opacity of kits were prepared by the IDS to be identical to ensure adequate blinding between placebo and BCAA dosages.

### Treatment

The three BCAAs, valine, isoleucine, and leucine, were combined together in a 1:1:1 ratio for the four treatment doses. Dose levels were determined both by animal models and prior clinical work in humans in other conditions. In brain-injured mice, the equivalent of 60 grams (g) per day of BCAAs mitigated cognitive impairment, without additional benefit at higher doses (Cole, et al. (2010) Proc. Natl. Acad. Sci., 107(1):366-371; Elkind, et al. (2015) Front Neurol., 6:73). In prior human trials, doses between 15 g and 57 g were utilized for treatment of neurologic conditions, such as tardive dyskinesia and hepatic encephalopathy (Als-Nielsen, et al. (2003) Cochrane Database Syst Rev. 2:CD001939; Richardson, et al. (2003) Am. J. Psychiatry 160(6):1117-1124). The “no observable adverse effect level” of BCAA intake is likely considerably higher than 60 g, with no evidence of toxicity in animal models up to 100 g/day and humans of up to 90 g per day (Tsubuku, et al. (2004) Int. J. Toxicol., 23(2):119-126). Given the unknown optimal dose of BCAA supplementation for concussion, doses of 15 g, 30 g, 45 g, and 60 g divided twice daily were chosen as initial treatment doses. However, during initial study preparation, precipitation issues were noted at the highest dose due to leucine precipitating at a lower concentration than expected. After further testing, it was determined that the product remained in solution at a concentration of 27 g/591 milliliter (mL) and, therefore, the highest dose level was altered to 54 g/day.

Each treatment dose was divided into twice daily dosing (7.5 g, 15 g, 22.5 g, and 27 g, respectively). For each dose, the BCAAs were combined and dissolved in water, with additives (sodium gluconate, Tropical Punch Kool-Aid® powder, and sucralose) in order to improve palatability. The placebo solution contained sucrose octaacetate and microcrystalline cellulose dissolved in water, with identical additives, to ensure the placebo solution had similar taste, texture, consistency, and appearance as the BCAA solution. BCAAs were supplied by Spectrum Chemical and Laboratory Products (New Brunswick, NJ).

### Monitoring and Outcomes

Participants were monitored for 21 days following enrollment. Participant baseline characteristics, including demographic data, was collected via self-report upon enrollment. Participants were monitored with daily electronic surveys as well as actigraphy as noted below; each daily survey included questions about concussion symptoms (including headache), physical activity, cognitive activity, drug compliance, adverse events, and a link to neurocognitive testing. In addition, in-person visits were conducted at three time points during study enrollment, between study days 5-9, 12-16, and 22-24.

The primary outcome was to determine if, compared to placebo treatment, BCAAs improved neurocognitive recovery. Neurocognitive testing was performed using the CogSport/Axon Sports Computerized Cognitive Assessment Tool (CCAT) (Maruff, et al. (2009) Arch. Clin. Neuropsychol., 24(2):165-178; Collie, et al. (2006) J. Neurol. Neurosurg. Psychiatry 77(2):241-245). The CCAT includes four computerized tests measuring processing speed (via a simple reaction time task), attention (via a choice reaction time task), visual learning (via a one card learning task) and working memory (via a one-back task). In line with studies and manufacturer recommendations, average log mean reaction time was compared at various time points for the processing speed, attention, and working memory subtests; accuracy was compared for the visual memory subtest (Maruff, et al. (2009) Arch. Clin. Neuropsychol., 24(2):165-178; Collie, et al. (2006) J. Neurol. Neurosurg. Psychiatry 77(2):241-245; Louey, et al. (2014) Arch. Clini. Neuropsychol., 29(5):432-441). Symptoms were assessed via self-report utilizing a 9-item symptom instrument, shown to have excellent factorial and construct validity (Piland, et al. (2006) Med. Sci. Sports Exerc., 38(1):27-32). Included in the symptom instrument are the following concussion-like symptoms: headache, nausea, balance problems, sleeping more than normal, drowsiness, fatigue, feeling slowed down, feeling mentally foggy, and difficulty concentrating. Each symptom is rated on a 7-point Likert scale (0=no symptom; 6=very severe symptoms), generating a total symptom score ranging from 0 to 54. Physical activity was rated on a 6-point Likert scale of return to baseline physical activity (0=no physical activity; 5=return to full physical activity, including full game play for athletes). Cognitive activity was rated on a 5-point Likert scale of return to baseline cognitive activity at school or work (0=complete cognitive rest; 4=full unrestricted participation in school or work).

Participant sleep was assessed using actigraphy, using the ActiSleep+™ Monitor (ActiGraph, Pensacola, FL) worn during the duration of participation on the participant's non-dominant wrist. Actigraphy was collected in 60 second epochs and analyzed using ActiLife® software version 6.13.4 with a 90-minute non-wear threshold (Knaier, et al. (2019) BMC Public Health 19(1):244; Choi, et al. (2012) Med. Sci. Sports Exerc., 44(10):2009-2016). Each study day was analyzed individually for bedtime, wake time, total sleep time (TST), sleep efficiency, wake after sleep onset (WASO), number and length of awakenings, activity, and proprietary indices of movement/sleep fragmentation. These respective metrics were averaged across the entire study duration, with daytime sleep periods excluded.

Participant compliance was assessed by self-report; as part of the daily survey, participants reported the number of bottles of study medication (0, 1, or 2, as each daily dose was divided into twice daily increments) that were consumed. For days in which participants did not answer the survey, it was assumed no drink was consumed. Potential adverse events, in accordance with the CONSORT harm guidelines for randomized trials, were collected from daily self-report surveys in addition to assessment at scheduled study visits. All adverse events were characterized with descriptions of the event, assessment of severity, indication of if the event was serious (death, a life-threatening event, hospitalization or prolongation of existing hospitalization, or persistent or significant disability/incapacity), and start and end date.

### Statistical Considerations and Power Analysis

Standard descriptive statistics were used to summarize demographic data. Kruskal-Wallis rank test was used to compare unadjusted measures for the four CCAT subtests, total symptom score, physical activity score, and cognitive activity score at day 7, 14, and 21 following enrollment among the placebo and four interventional arms in an intention-to-treat approach. The initial analytic plan was to adjust for age and repeated measures using a mixed effects model by prescribed dose; given both the inability to reach the enrollment goal, as well as varying response rates (see Table 1), it was not possible to fit a model using this approach. Instead, to account for individual characteristics, such as age and varying amounts of follow-up time with unbalanced numbers of repeated measures, in addition to capturing efficacy as opposed to effectiveness (given varying adherence to treatment protocol, see Table 3), a linear regression was used to conduct a dose-response analysis. The linear regression model included total cumulative study dose received (study dose prescribed multiplied by number of days consuming said dose) as the independent variable and change in outcome (average of each participant's first three measures compared with the participant's last three measures over the course of the study) as the dependent variable among participants with at least seven reported measurements. This dose-response model was fitted separately for each of the four CCAT subtests, total symptom score, physical activity, and cognitive activity; the intercept and slope parameter (with 95% confidence interval and p value) were obtained from models with each outcome. Although both physical and cognitive activity were assessed on ordinal scales, the outcome measure used in the model was the change in these activity levels, leading to a continuous variable from which a linear regression model could be fitted. Sleep data was analyzed using two-tailed students t-tests across groups, with the primary comparison being placebo vs. 54 g/day due to relative group sample sizes for this outcome. Compliance (average reported drink consumed over study period) and adverse events (average per arm and percent in each arm experiencing adverse events) were compared across arms using Kruskal-Wallis rank test and Fisher's exact test, where appropriate. Analyses were performed using SAS software, version 9.4 (SAS Institute Inc, Cary, NC), Stata software, release 14.2 (StataCorp LLC, College Station, TX), and GraphPad Prism version 8 (GraphPad Software, San Diego, CA).

TABLE 1
Participant demographics.

| | | 15 g | 30 g | 45 g | 54 g | |
|---|---|---|---|---|---|---|
| | Placebo | BCAA | BCAA | BCAA | BCAA | p- |
| | (n = 8) | (n = 8) | (n = 5) | (n = 9) | (n = 8) | value |
| Age in years, median | 16.5 | 15 | 17 | 15 | 15.5 | 0.937 |
| (IQR) | (14.5, 18.5) | (15, 16.5) | (15, 17) | (15, 16) | (13.5, 21) | |
| Female sex, n(%) | 4 | 4 | 2 | 4 | 4 | 1.000 |
| | 50.0% | 50.0% | 40.0% | 44.4% | 50.0% | |
| Race/Ethnicity, n(%) | | | | | | 0.850 |
| Non-Hispanic White | 6 | 6 | 4 | 7 | 7 | |
| | 75.0% | 75.0% | 80.0% | 77.8% | 87.5% | |
| Non-Hispanic Black | 1 | 1 | 0 | 2 | 0 | |
| | 12.5% | 12.5% | 0% | 22.2% | 0.0% | |
| Hispanic | 1 | 0 | 1 | 0 | 1 | |
| | 12.5% | 0.0% | 20.0% | 0.0% | 12.5% | |
| Asian-American | 0 | 1 | 0 | 0 | 0 | |
| | 0.0% | 12.5% | 0.0% | 0.0% | 0.0% | |
| Days from injury to | 2 | 2 | 2 | 2 | 2.5 | 0.921 |
| enrollment, median (IQR) | (1.5, 2.5) | (2, 2.5) | (2, 2) | (2, 2) | (1.5, 3) | |
| Total symptom score at | 12 | 18 | 6 | 28.5 | 27 | 0.336 |
| enrollment, median (IQR) | (8, 29) | (6, 30) | (4, 43) | (19, 34) | (25, 38) | |
| Median dose consumed | 0 | 405 | 600 | 450 | 1107 | 0.001 |
| across study (g, IQR) | (0.0) | (157.5, 555 | (180, 690) | (225, 765) | (513, 1647) | |
| Median number days | 17.5 | 15.5 | 13 | 5 | 14.5 | 0.282 |
| survey answered (IQR) | (8.5, 20.5) | (5.5, 19) | (10, 13) | (4, 11) | (7.5, 17) | |
| Completed | 6 | 5 | 4 | 8 | 7 | 0.687 |
| neurocognitive testing, | 75.0% | 62.5% | 80.0% | 88.9% | 87.5% | |
| n(%) | | | | | | |
| Median number days | 17.5 | 16 | 12 | 6 | 13 | 0.633 |
| neurocognitive testing | (2, 20) | (2, 18) | (10, 14) | (2, 12.5) | (5, 16) | |
| completed | | | | | | |
| Enrolled 2014-2016, | 2 | 3 | 1 | 3 | 3 | 1.000 |
| n(%) | 25.0% | 37.5% | 20.0% | 33.3% | 37.5% | |
| Enrolled 2018-2020, | 6 | 5 | 4 | 6 | 5 | |
| n(%) | 75.0% | 62.5% | 80.0% | 66.7% | 62.5% | |

BCAA = branched chain amino acid.
IQR = interquartile range.

Prior to study initiation, an enrollment goal of 50 subjects, or 10 subjects per arm, was anticipated, which would have given 80% power to detect differences in the processing speed subtest between the BCAA arms and the placebo arm of 0.122 seconds (mean log −0.914), or a 5% difference based on healthy control data (including a standard deviation of 0.08 seconds in healthy individuals, inflated to 0.10 for injured subjects) (Louey, et al. (2014) Arch. Clini. Neuropsychol., 29(5):432-441). As noted above, due to significant slowing of enrollment during the novel coronavirus pandemic in early 2020, in late 2020, after randomizing 42 participants, study enrollment was halted to assess efficacy of data.

### RESULTS

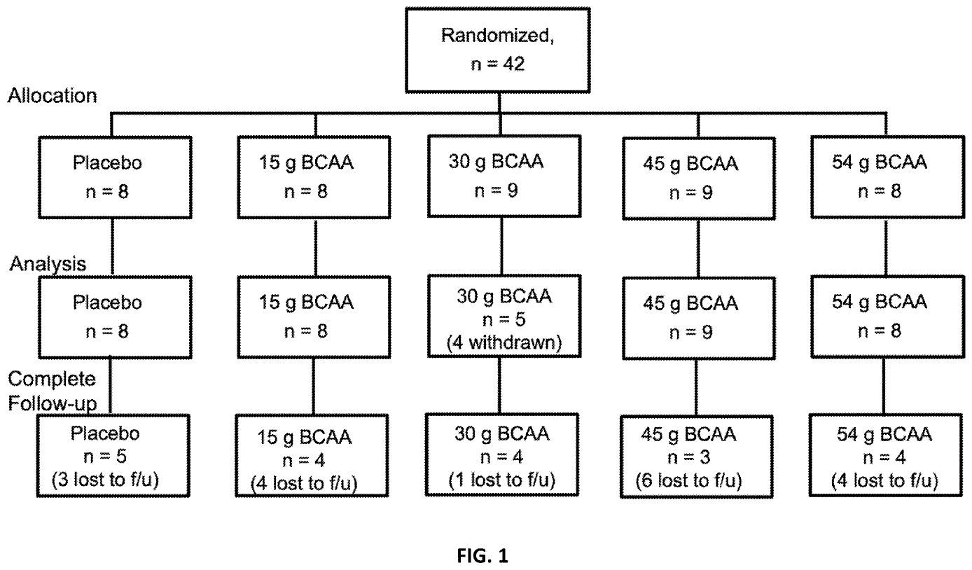

In total, 42 participants were randomized (see FIG. 1 for flow diagram). Of those, four participants did not provide any follow-up data (all in the 30 g BCAA arm), and therefore were withdrawn from further analysis. Demographics and initial injury characteristics for the 38 analyzed participants are provided in Table 1. Of the 38 participants analyzed, 26 provided at least seven reported measures in symptoms, physical and cognitive activity, and 18 provided at least seven reported measures for neurocognitive testing; 20 provided data through the full 21 days of the study period. Average number of days for which the survey questions and CCAT battery were completed across the study period by arm is displayed in Table 1. Overall, the median total study dose consumed was 360 g (range 0-2052 g; see Table 1 for breakdown by arm).

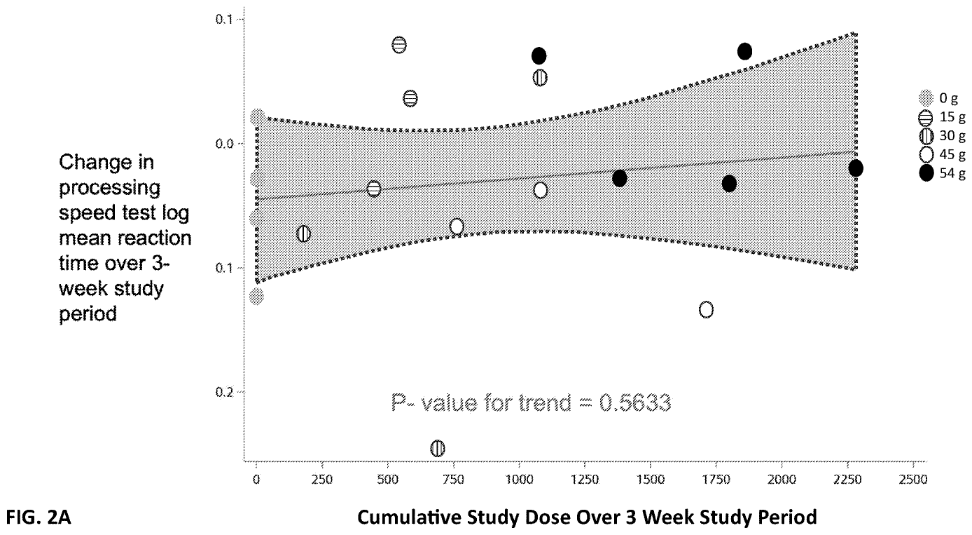

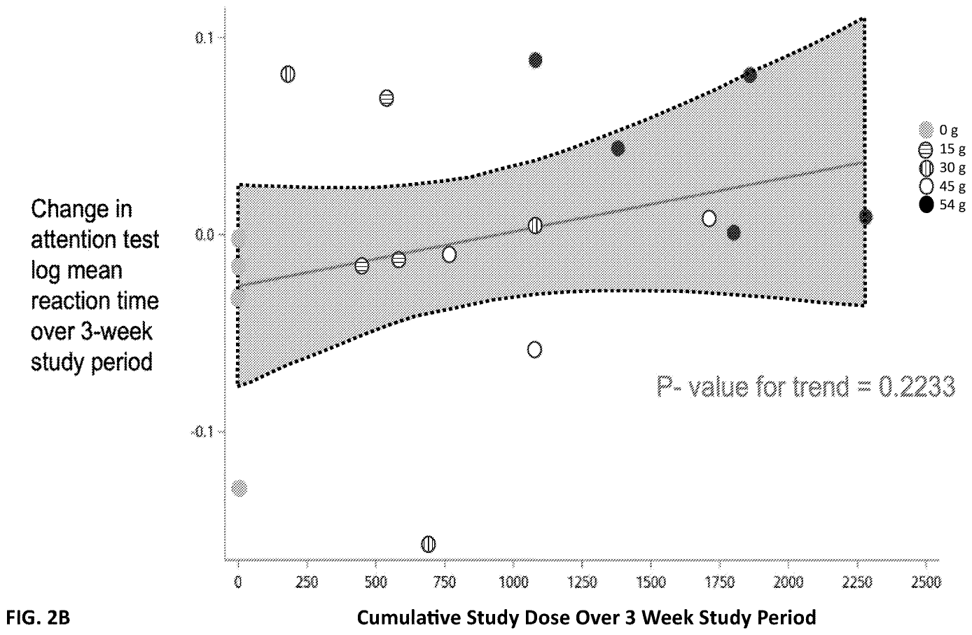

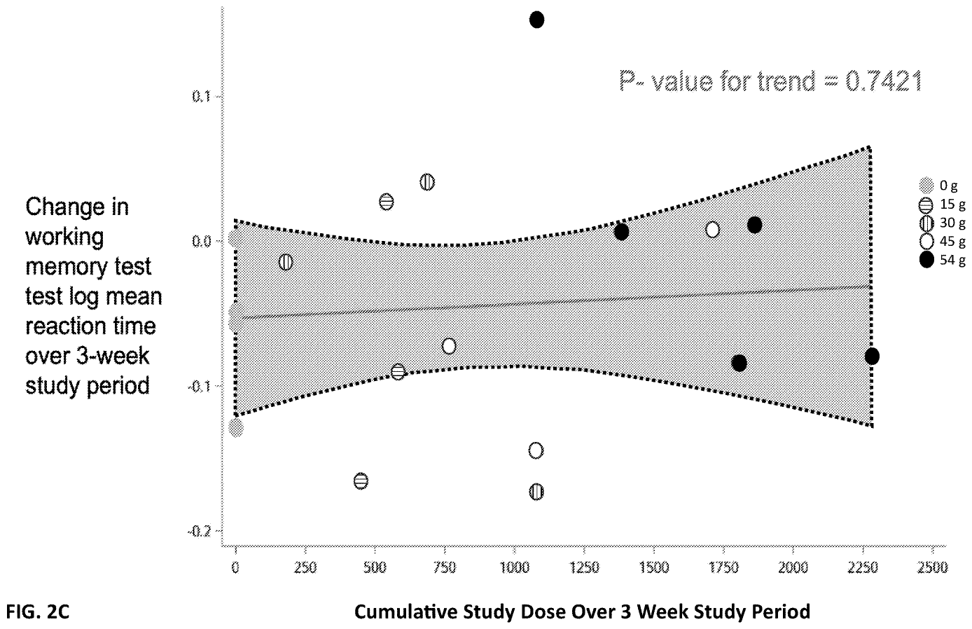

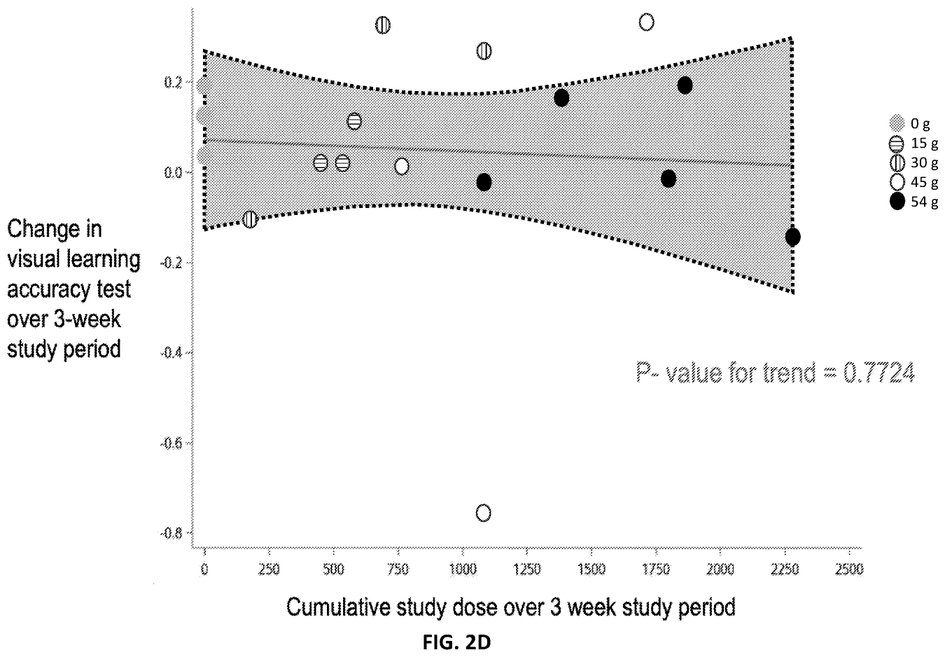

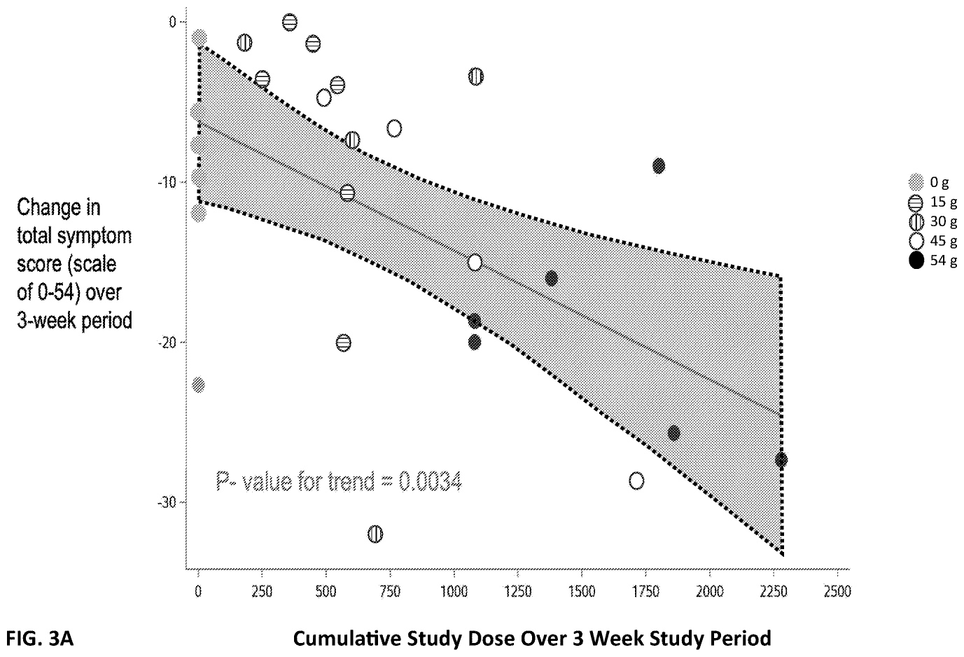

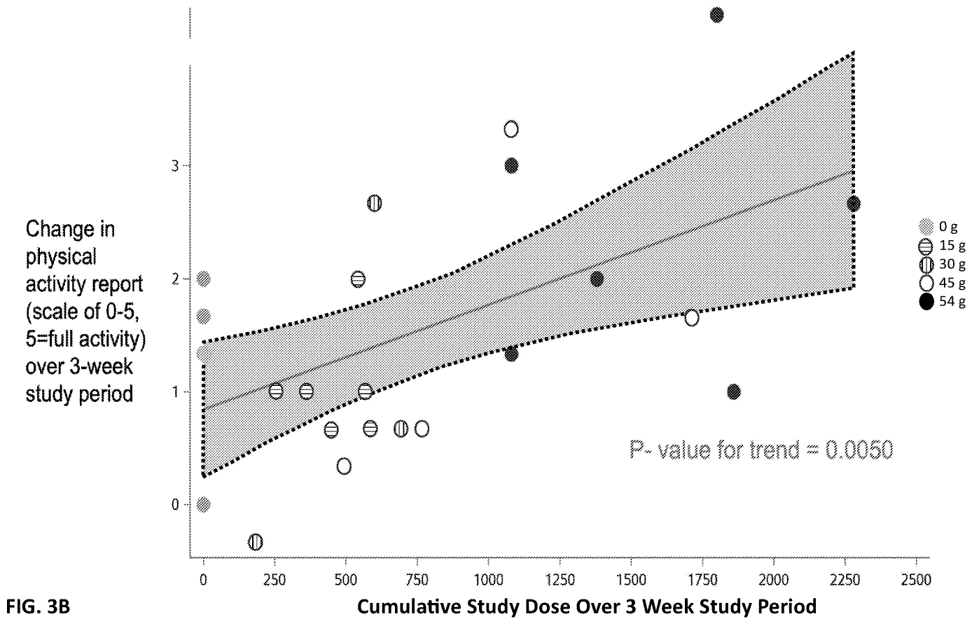

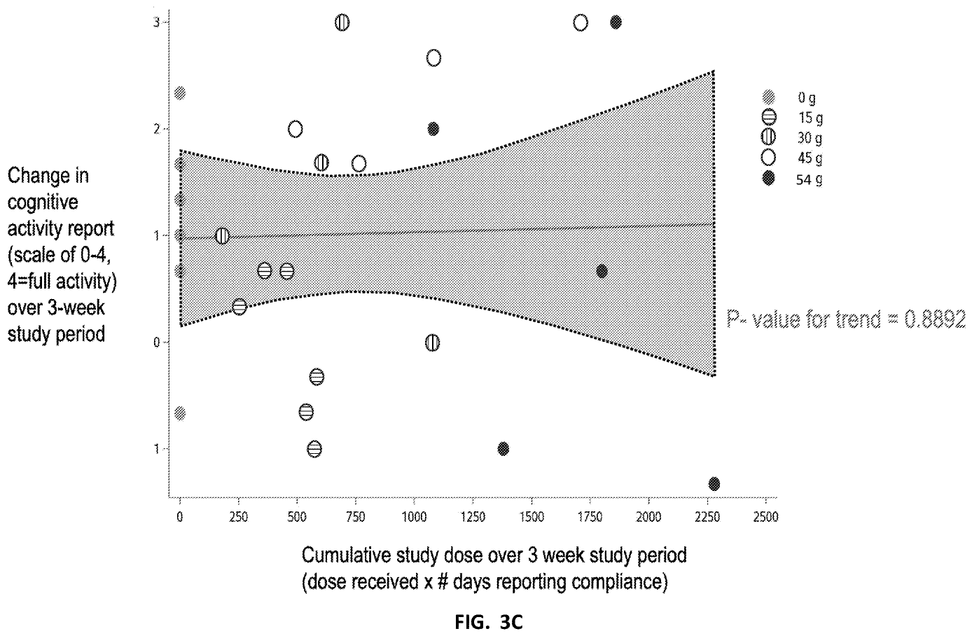

In terms of neurocognitive testing, no differences were found in reaction time for the processing speed, attention, and working memory subtests, nor in accuracy for the visual memory subtest, in either the intention-to-treat unadjusted analysis or the adjusted per-protocol dose-responses analysis (Table 4; FIGS. 2A-2D). Of note, not all patients who completed follow-up completed the neurocognitive testing elements (Table 1). A significant reduction in total symptom score by total dose consumed using was found using a linear regression analysis (a decrease of 4.4 points [standard error 1.4] for each 500 g of study drug consumed, p value for trend line=0.0036; Table 4 and FIG. 3A), as well as a significant improvement in return to baseline physical activity by total dose consumed in the adjusted analysis (an increase of 0.503 points [standard error 0.164] for each 500 g of study drug consumed, p value for trend=0.0053; FIG. 3B). No change in cognitive score was found in either the unadjusted or adjusted (FIG. 3C) analyses.

With respect to sleep outcomes, actigraphy analyses demonstrated significantly improved sleep efficiency (p=0.036) and total sleep time (p=0.042) in the placebo vs. 54 g/day comparison (Table 2). No statistically significant differences across groups were observed for sleep efficiency, total sleep time and WASO across groups, although trends were present (e.g., p-values ranging between 0.072-0.091). Notably, the raw values for sleep efficiency (93.2% vs. 91.4%) and total sleep time (536.7 minutes vs. 468.1 minutes) were higher in the highest BCAA dose group (54 g/day) versus the placebo group.

TABLE 2
Sleep measures collect by actigraphy. Data are
mean +/− standard deviation.

| | | 15 | 30 | 45 | 54 |
|---|---|---|---|---|---|
| | Placebo | g/day | g/day | g/day | g/day |
| | n = 5 | n = 6 | n = 3 | n = 3 | n = 6 |
| Bedtime, | 23:44 | 22:59 | 23:37 | 23:28 | 22:52 |
| hh:mm | | | | | |
| Waketime, | 7:46 | 7:17 | 8:10 | 8:08 | 7:25 |
| hh:mm | | | | | |
| Efficiency, | 91.4 ± | 90.7 ± | 93.4 ± | 90.4 | 93.2 ± |
| % | 4.3 | 13.0 | 3.1 | 4.7 | 4.1* |
| TST, min | 468.1 ± | 496.0 ± | 488.1 ± | 474.3 ± | 536.7 ± |
| | 93.8 | 143.0 | 125.7 | 150.5 | 132.5* |
| WASO, min | 41.0 ± | 47.6 ± | 30.5 ± | 44.3 ± | 42.0 ± |
| | 22.6 | 35.4 | 14.9 | 23.8 | 29.5 |
| # of | 17.9 ± | 18.6 ± | 12.6 ± | 18.5 ± | 16.0 ± |
| Awakenings | 8.5 | 9.6 | 6.4 | 9.3 | 9.6 |
| Length of | 2.4 ± | 2.6 ± | 2.6 ± | 2.8 ± | 2.6 ± |
| Awakenings, | 1.1 | 1.5 | 1.2 | 1.4 | 1.6 |
| min | | | | | |
| Activity | 20858.3 ± | 22446.5 ± | 16491.5 ± | 24245.0 ± | 20365.7 ± |
| Counts | 9383.7 | 11301.7 | 8015.2 | 11531.1 | 12805.4 |
| Movement | 15.3 ± | 15.5 ± | 10.4 ± | 14.7 ± | 12.8 ± |
| Index | 6.3 | 7.3 | 4.7 | 7.1 | 8.3 |
| Sleep | 29.3 ± | 26.1 ± | 25.9 ± | 27.4 ± | 23.9 ± |
| Fragmenta- | 13.7 | 13.5 | 11.1 | 14.9 | 18.6 |
| tion Index | | | | | |

*p = <0.05 vs. placebo. TST = total sleep time. WASO = wake after sleep onset.

In analyzing the compliance, a non-significant decrease in average reported total drink consumed by arm was found (81% reported compliance in the placebo arm compared to 49% compliance in the 54 g BCAA arm; Table 3). However, no difference in adverse events by arm was found (Table 3). Across the study, in total, 13 adverse events were reported among the 38 participants. None were severe, and the majority (10 out of 13, including all three adverse events in the 30 g 45 g and 54 g BCAA arms) were reported as mild. The majority (12 of the 13) were gastrointestinal side effects (abdominal pain, diarrhea, or bloating), with higher numbers of adverse events reported per participant in the placebo (0.63) and 15 g BCAA (0.63) arms compared to the higher dose BCAA arms (0.20, 0.00, and 0.25).

TABLE 3
Compliance and adverse events.

| | | 15 g | 30 g | 45 g | 54 g | |
|---|---|---|---|---|---|---|
| | Placebo | BCAA | BCAA | BCAA | BCAA | p- |
| | (n = 8) | (n = 8) | (n = 5) | (n = 9) | (n = 8) | value |
| Percent total drink | 81.0 | 64.3 | 54.8 | 23.8 | 49.0 | 0.581 |
| consumed, median (IQR) | (14.3, 87.9) | (28.4, 91.4) | (15.0, 55.6) | (11.9, 42.5) | (22.7, 72.6) | |
| Total adverse events, n | 5 | 5 | 1 | 0 | 2 | |
| Participants reporting | 3 | 3 | 1 | 0 | 1 | 0.203 |
| adverse event, n(%) | 37.5% | 37.5% | 20% | 0% | 12.5% | |
| Number of adverse | 0.63 | 0.63 | 0.20 | 0.00 | 0.25 | |
| events per participant in | | | | | | |
| study arm | | | | | | |

BCAA = branched chain amino acid.
IQR = interquartile range.

TABLE 4
Distribution of measures of change in outcomes (calculated as
the difference of the average of the first three measurements
and last three measurements among participants with seven or
more measurements), the change and standard error of change,
and the correlation with total cumulative study dose.

| | | | | Change in |
|---|---|---|---|---|
| | | Mean | | outcome |
| | Number of | change | Median | for increase |
| | participants | (standard | change | of total study |
| | evaluated | deviation) | (range) | dose of 500 g |
| Processing | 18 | −0.031 | −0.031 | 0.008 |
| speed | | (0.083) | (−0.246, | |
| | | | 0.080) | |
| Attention | 18 | −0.002 | −0.001 | 0.014 |
| | | (0.066) | (−0.158, | |
| | | | 0.089) | |
| Working memory | 18 | −0.045 | −0.052 | 0.004 |
| | | (0.083) | (−0.173, | |
| | | | 0.153) | |
| Visual learning | 18 | 0.050 | 0.076 | −0.013 |
| | | (0.242) | (−0.757, | |
| | | | 0.332) | |
| Total symptom | 26 | −12.1 | −9.3 | −4.4 |
| score | | (9.6) | (−32, 0) | |
| Physical | 26 | 1.51 | 1.33 | 0.503 |
| activity score | | (1.14) | (−0.33, | |
| | | | 4.33) | |
| Cognitive | 26 | 1.01 | 1.00 | 0.051 |
| activity score | | (1.32) | (−1.33, | |
| | | | 3.00) | |

| | Standard | Pearson | p value for |
|---|---|---|---|
| | error for | correlation | increase in |
| | increase in | coefficient with | total study |
| | total study | total cumulative | dose change |
| | dose of 500 g | study dose | of 500 g |
| Processing speed | 0.016 | 0.124 | 0.6238 |
| Attention | 0.012 | 0.283 | 0.2554 |
| Working memory | 0.0016 | 0.065 | 0.7964 |
| Visual learning | 0.046 | −0.073 | 0.7749 |
| Total symptom score | 1.4 | −0.550 | 0.0036 |
| Physical activity score | 0.164 | 0.531 | 0.0053 |
| Cognitive activity | 0.224 | 0.046 | 0.8234 |
| score | | | |

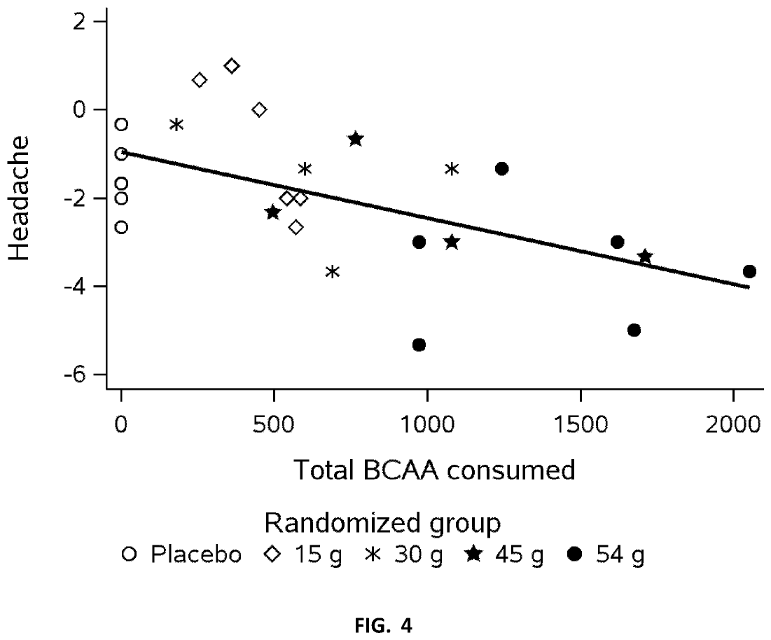

As stated hereinabove, a significant reduction in total symptom score by total dose consumed was found. Surprisingly, a very significant reduction in headache was observed in subjects consuming the BCAAs compared to placebo (see Table 5 and FIG. 4). More specifically, a significant reduction was found in the presence and the severity of self-reported headache by total dose consumed using linear regression analysis, a decrease of 0.75 points [standard error 0.22] on the 0-6 scale for each 500 g of study drug consumed, p-value for trend line=0.0020.

TABLE 5
Distribution of measures of change in headache,
the change and standard error of change, and the
correlation with total cumulative study dose.

| | | | | Change in outcome |
|---|---|---|---|---|
| | Number of | Mean change | Median | for increase of |
| | participants | (standard | change | total study dose |
| | evaluated | deviation) | (range) | of 500 g |
| Headache | 26 | −1.99 | −2.00 | −0.752 |
| score | | (1.57) | (−5.33, | |
| (scale 0-6) | | | 1.00) | |

| | | Pearson | |
|---|---|---|---|
| | Standard error | correlation | p-value for |
| | for increase | coefficient with | increase in |
| | in total study | total cumulative | total study dose |
| | dose of 500 g | study dose | change of 500 g |
| Headache | 0.217 | −0.577 | 0.0020 |
| score | | | |
| (scale 0-6) | | | |

In summary, this double-blinded, randomized controlled trial, the first evaluation of BCAA supplementation in the treatment of concussion in adolescents and young adults, demonstrated efficacy, high tolerability, and safety of BCAAs. Specifically, a significant dose response effect in reduction of concussion symptoms (particularly headache), improvement in physical activity, and improvement in sleep measures were found with those who consumed higher total doses of BCAAs across the study period, with high tolerability of treatment doses without serious adverse events.

BCAAs participate directly and indirectly in a variety of important biochemical functions in the brain. In addition to the fundamental use of amino acids for protein synthesis, these specific essential amino acids are pivotal in glutamate synthesis, as they contribute 50% of the nitrogen in glutamate (Sakai, et al. (2004) J. Neurochem., 88(3):612-622). Moreover, the decarboxylation of glutamate by glutamic acid decarboxylase (GAD) leads to the synthesis of the primary inhibitory neurotransmitter GABA. Furthermore, de novo glutamate synthesis contributes a significant amount (approximately 40%) of releasable synaptic glutamate (Lieth, et al. (2001) J. Neurochem., 76(6):1712-1723). It has been hypothesized that the increased energy demands of the brain post-injury rapidly deplete BCAA levels (Cole, et al. (2010) Proc. Natl. Acad. Sci., 107(1):366-371). In addition, it has been noted that BCAAs can enter the Krebs cycle and contribute to increased production of adenosine triphosphate (ATP), alterations in which have also been implicated as a key component to the pathophysiology of concussion (Giza, et al. (2014) Neurosurgery 75(0 4):S24-S33; Aquilani, et al. (2005) Arch. Phys. Med. Rehabil., 86(9):1729-1735). Interestingly, animal studies have found that repeated exposure to BCAAs over multiple days is required to observe improvements in cognitive function following TBI (Elkind, et al. (2015) Front Neurol., 6:73). This indicates that it is not only BCAA supplementation, but repeated exposure to BCAAs, that underlies the clinical improvement noted in the current study as evidenced by the total dose-response effect in improving concussion symptoms and activity levels following injury.

Overall, minimal adverse effects with BCAA supplementation, particularly at the higher doses, was observed. Multiple studies have reported the tolerability of BCAAs, with large reviews reporting either no or minimal gastrointestinal side effects, and the results herein further support the tolerability of BCAA supplementation.

The improvement in concussion-related symptoms, specifically headache as the most common concussion symptom observed acutely and in follow-up visits (Eisenberg, et al. (2013) Pediatrics 132(1):8-17; Blinman, et al. (2009) J. Pediatr. Surg., 44(6):1223-1228), as well as physical activity, in a clear dose-response pattern is a key finding of the study. Symptoms (including headache) have emerged as the principal primary outcome in the largest observational and interventional trials of concussion (Leddy, et al. (2021) Lancet Child Adolesc. Health 5(11):792-799; Zemek, et al. (2016) JAMA 315(10):1014-1025). In addition, there has been a push to incorporate patient-reported outcomes as primary measures of functioning following injury (with return to baseline activity a key component of post-injury functioning) and a strong correlation between symptom burden and quality of life in adolescent concussion has been demonstrated (Fineblit, et al. (2016) J. Neurotrauma 33(17):1561-1568; Howell, et al. (2019) Clin. Pediatr., 58(1):42-49). While the primary outcome, neurocognitive testing, did not show significant differences among the treatment arms, since the inception of the study, multiple limitations to utilizing neurocognitive testing as an objective injury marker have been described (Hang, et al. (2015) Pediatr. Emerg. Care 31(10):688-693; McClure, et al. (2014) Am. J. Sports Med., 42(2):472-478). In particular, a “ceiling effect” to neurocognitive testing has been described, reducing its utilization as a study endpoint over time (Gaudet, et al. (2021) Arch. Clin. Neuropsychol., 36(4):561-569). Since the initial development of the study protocol, however, multiple other objective markers of injury have been developed as key metrics of central nervous system dysfunction, including visio-vestibular testing, evaluation of the pupillary light reflex, and addition to blood-based biomarkers (Corwin, et al. (2020) Med. Sci. Sports Exerc., 52(3):542-548; Corwin, et al. (2015) J. Pediatr., 166(5):1221-1225; Master, et al. (2020) JAMA Ophthalmol., 138(11):1135-1141; Mannix, et al. (2020) J. Neurotrauma 37(19):2029-2044).

Sleep disturbances have frequently been reported as key elements contributing to dysfunction following concussion, with sleep alterations in the acute time frame after injury having been identified as a key prognostic factor in the development of persistent post-concussive symptoms (Mosti, et al. (2016) Neurol. Clin. Pract., 6(2):129-137; Tang, et al. (2022) Neurotrauma Rep., 3(1):276-285). The efficacy of BCAA supplementation to improve sleep disturbances in animal models has been shown and a recent randomized trial of veterans with chronic TBI symptoms showing BCAA efficacy in improving both subjective and objective sleep impairment (Lim, et al. (2013) Sci. Transl. Med., 5(215):215ra173; Elliott, et al. (2022) Front. Syst. Neurosci., 16:854874). The present data supports this conclusion. The results demonstrate a statistically significant improvement in sleep efficiency and total sleep time between placebo and the 54 g/day group, supporting BCAA supplementation as a therapeutic means to improve sleep in acute concussion in the adolescent and young adult population, a critical finding given the implications of insufficient sleep in this population (Owens, et al. (2014) Pediatrics 134(3):e921-e932).

Minimal adverse effects with BCAA supplementation were observed, particularly at the higher doses. The tolerability of BCAAs has been reported, with large reviews reporting either no, or minimal gastrointestinal, side effects, and the results herein further support the tolerability of BCAA supplementation (Als-Nielsen, et al. (2003) Cochrane Database Syst Rev., 2:CD001939).

Several randomized trials of active rehabilitative treatment programs have been conducted showing efficacy in reducing prolonged concussion symptoms (Leddy, et al. (2019) JAMA Pediatr., 173(4):319-325; Leddy, et al. (2021) Lancet Child Adolesc. Health 5(11):792-799; Kontos, et al. (2021) J. Pediatr., 239:193-199). However, these interventions require either in-person visits or specialized guidance to complete (Corwin, et al. (2021) Pediatr. Emerg. Care 37(7):371-379). The potential reliance on specialist prescription and monitoring has the potential to introduce or exacerbate known disparities in concussion care that exist for underserved communities (Mohammed, et al. (2023) J. Head Trauma Rehabil., 38:147-155). This underlies the importance of developing safe, well-tolerated pharmacologic interventions for concussion in adolescents and young adults, such as BCAA supplementation, that could be prescribed from diverse settings without a need for specialist mentoring. The role of BCAAs may also expand beyond treatment into the realm of injury prevention, as a recent animal model study explored the efficacy of administering BCAAs in mice prior to head trauma as a prophylaxis, with improved motor recovery and cognitive function (Dickerman, et al. (2022) Neurotrauma Rep., 3(1):321-332).

In conclusion, this study demonstrates the efficacy of BCAAs in improving concussions symptoms (particularly headache); more rapidly allowing a return to baseline activity; and improving sleep measures in concussed adolescents and young adults, the first such data in acute mild traumatic brain injury in humans. Importantly, BCAAs were found to be well-tolerated with minimal adverse events, even at the highest doses, in the participants. These data show the benefit of BCAA supplementation to reduce symptoms and increase activity following concussion.

A number of publications and patent documents are cited throughout the foregoing specification in order to describe the state of the art to which this invention pertains. The entire disclosure of each of these citations is incorporated by reference herein.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1: A method for reducing a pathology associated with traumatic brain injury in a subject in need thereof, said method comprising administering to said subject at least one branched chain amino acid.

2: The method of claim 1, wherein said traumatic brain injury is an acute, mild traumatic brain injury.

3: The method of claim 1, wherein said traumatic brain injury is a concussion.

4: The method of claim 1, wherein said method comprises administering valine, leucine, and isoleucine to said subject.

5: The method of claim 1, wherein said branched chain amino acid is in a composition with at least one pharmaceutically acceptable carrier.

6: The method of claim 1, wherein said subject is less than 35 years of age.

7: The method of claim 1, wherein said pathology is a headache.

8: The method of claim 1, wherein said pathology is reduced physical activity.

9: The method of claim 1, wherein said pathology is reduced sleep efficiency and/or sleep time.

10: The method of claim 1, further comprising administering acetate to said subject.

11: The method of claim 1, wherein said branched chain amino acids are administered orally.

12: The method of claim 1, wherein said method comprises administering to said subject a composition comprising valine, leucine, isoleucine, a flavor masking agent, a sweetener, and a flavoring agent.

13. (canceled)

14: The method of claim 12, wherein said flavor masking agent is a gluconate salt.

15: The method of claim 12, wherein said sweetener is sucralose.

16: The method of claim 1, wherein said branched chain amino acid is administered for at least 10 consecutive days.

17: The method of claim 1, wherein said method comprises administering at least about 50 g of valine, leucine, and isoleucine per day.

18: The method of claim 12, wherein said composition comprises valine, leucine, isoleucine, a flavor masking agent, and a sweetener in a ratio of about 1:1:1:1.3:0.9.

19: The method of claim 12, further comprising administering acetate to said subject.

20. (canceled)

21: A method for treating a headache in a subject in need thereof, said method comprising administering to said subject at least one branched chain amino acid, wherein said subject has a traumatic brain injury.

22-38. (canceled)

39: The method of claim 21, wherein the duration, severity, and/or presence of the headache is reduced.
